(19) United States
(12) Patent Application Publication
(10) Pub. No.: US 2003/0125783 A1
(43) Pub. Date: Jul. 3, 2003

(54) Device and method for wound healing and debridement

(75) Inventor: Kelly Moran, Wilbraham, MA (US)

Correspondence Address: BOLESH J. SKUTNIK PhD, JD, 515 Shaker Road, East Longmeadow, MA, 01028, US

(73) Assignee: CeramOptec Industries, Inc.

(21) Appl. No.: 10/022,989

(22) Filed: Dec. 18, 2001

(51) Int. Cl. A61N 5/067

(52) U.S. Cl. 607/89

(57) ABSTRACT

A method and device is disclosed for wound debridement and wound healing that is equally effective for both diabetic and non-diabetic patients. A non-ablative laser beam or non-coherent radiation of a wavelength between 193 nm and 10.6 microns, preferably between 400 and 1350 nm, is applied in combination with a mechanical or laser debriding apparatus. The radiation is delivered at a power density of at least approximately 1 W/cm² over a time period ranging from 1 second to 3 minutes. Typically, radiation power is in the range of 1 Watt to 15 Watts. Power density and treatment duration are adjusted to the parameters of the individual wound. Delivery means include, but are not limited to, optical fibers, articulating arms or direct exposure to radiation-emitting sources. In a preferred embodiment, pulsed or continuous wave high power radiation of an appropriate wavelength and a laser or mechanical scraping apparatus are intermittently or simultaneously applied to a wound. This device can be used on wounds of various depths to drastically cut down on the healing time by killing viral bodies and bacteria and performing other tasks such as vascularization.

### BACKGROUND OF THE INVENTION

1. Field of the Invention

This invention relates to the use of electromagnetic radiation in a method and device that combines wound debridement and wound healing.

2. Information Disclosure Statement

There are a wide variety of techniques currently used for wound debridement and an equally wide variety of techniques for stimulating wound healing. Wound debridement is more particularly described as the surgical removal of foreign material and/or dead, damaged and infected tissue from a wound to expose healthy tissue. Debridement is utilized to aid in the healing process and prevent infection. Techniques for debridement include autolytic and enzymatic debridement (use of enzymes or the body's naturally produced fluids), mechanical debridement (wet-to-dry dressings, hydrotherapy, irrigation), and surgical debridement.

Wound healing refers to those techniques used to stimulate the wound bed or surrounding tissue to accelerate regrowth. Electromagnetic radiation has been shown to have a beneficial effect on wound healing rates, and such techniques can often be used after a wound has been debrided.

Low level laser therapy (LLLT), or the use of low power lasers for therapeutic treatment, is a technique currently used to promote wound healing. LLLT devices typically deliver 10 mW-200 mW of power during treatment. Power densities typically range from 0.05 W/Cm²-5 W/cm², and most LLLT treatments deliver energy densities in the range of 0.5 -10 J/cm². This technique utilizes a process termed photonic biostimulation. It is claimed that exposing a wound to visible and infrared radiation produces observable reductions in the amount of time required for a wound to heal.

An example of LLLT is described in U.S. Pat. No. 5,766,233 by Thiberg, which utilizes a light-emitting diode array to deliver infrared radiation and visible red light. Pulsed infrared radiation is emitted by a large array of individual low power diodes that emit a combined total power of 900 mW over 3 minutes, followed by pulsed visible red light emitted by individual low power diodes that together produce a combined total intensity of about 3000 millicandela, with an associated power of approximately 900 mW, over 3 minutes. This invention emphasizes the need to irradiate the treatment site with both visible and infrared radiation. Furthermore, the array of low power diodes creates a beam size with a large surface area, which results in a low power density.

U.S. Pat. No. 5,259,380 (Mendes et al.) discloses a low power laser therapy system consisting of a focused array of light-emitting diodes. It specifically discloses an array for emitting visible red light for wound healing with a power density on the order of 15 mW/cm², utilizing powers between 2 and 10 mW and treatment times ranging from 7 to 20 minutes. The maximum energy density prescribed in this invention, using the prescribed power density over a 7 minute period, is approximately 6.3 J/cm².

U.S. Pat. No. 6,267,779 discloses an apparatus for biostimulation and treatment of tissue consisting of two focusable laser treatment wands for the continuous or pulsed emission of coincident infrared and visible radiation. The patent prescribes a power range of 0-2 Watts, and specifies an energy range of 1-99 Joules over treatment durations ranging from 1-60 minutes. The patent claims that the intersection of the beams in the patient's body has an increased therapeutic effect.

U.S. Pat. No. 5,445,146 discloses a method for pain reduction and healing using a low level reactive laser system operating at 1064 nanometers and delivered with power between 100 and 800 mW. The applied energy density is limited to the range of 1-15 J/cm². U.S. Pat. No. 5,951,596, a continuation-in-part of the above patent, discloses a similar method using a laser operating at 1,000-1,150 nm. The laser is delivered with a power density of 100-1,000 mW/cm² and an energy density of 1-15 J/cm². Both of the above patents teach the use of low power lasers to stimulate soft tissue with the aim of reducing pain and inflammation and stimulating microcirculation to enhance healing. Radiation is applied that is sufficient to elevate the temperature of the treated tissue, but is less than that required to convert tissue into a collagenous substance. These methods do not perform other functions such as stimulating regrowth or vascularization, and they specifically restrict the applied power levels to avoid such results.

Though there are many low level laser therapy applications for a variety of medical applications, there is currently no conclusive proof of LLLT's usefulness in stimulating tissue repair and wound healing. LLLT applications' lack of effectiveness arises from the use of lower radiation powers over long periods of time.

It has been shown that the use of high powered 980 nm lasers are quite effective in accelerating wound healing, as is seen in U.S. Pat. No. 6,165,205 by Neuberger. Lasers with a wavelength of 980 nm dosed in high power—5 Watts compared to 250 mW typical in many LLLT applications—have unique properties, as seen in empirical studies done in the context of wound therapy. Studies have shown that 980 nm lasers have the capability to stimulate tissue growth and vascularization, and to kill bacteria and viral bodies. In addition, the laser is able to do this without causing pain or discomfort in the patient, or at least with a minimum of pain or discomfort. Lasers of this wavelength are also capable of being used as cutting instruments, and have the ability to simultaneously cut and cauterize the resulting wound, thus reducing bleeding. These characteristics are beneficial to both the patient and surgeon, and are of particular benefit to diabetic patients. However, this invention is limited to a 980 nm wavelength laser.

It would be beneficial to utilize other wavelengths in a method that is effective for wound healing stimulation. For example, a laser in the low infrared range, which is within the light spectrum documented as having an optimum biological effect on tissue and having maximum penetration at low powers, could also be used with higher powers to enhance its therapeutic effect. The beneficial effects of 830 nm wavelength radiation are described by Smith in U.S. Pat. No. 5,464,436. The invention utilizes a complex three-step process. The first step involves diagnosing an afflicted area and delivering to that area a very low power dose of laser light with a wavelength between 800-870 nm, preferably with a wavelength of 830 mn. The radiation is delivered with an energy density of 1 J/cm2 over a preferably 33 second duration. The second step involves monitoring the treatment site. The third step involves repeating the diagnosis and treatment based on the monitoring step.

Sharp surgical and laser surgical debridement techniques are extremely effective for the selective removal of necrotic or infected tissue, and are especially effective for wounds with a large amount of necrotic, or dead, tissue. They are the fastest wound debridement techniques, and are also the most selective, giving the surgeon superior control over what tissue is to be removed. Unfortunately, these techniques are often extremely painful and can be costly. Improvements have been made on debriding techniques intended primarily for the removal of damaged or destroyed skin as a result of severe burns. Pulsing CO₂ lasers are used to debride burn wounds by ablating the eschar (hardened, black tissue) of a burn wound.

Many current applications focus on using the laser for cutting away dead, damaged and infected tissue, in essence using the laser in the same way a scalpel is used. This is an unnecessary procedure, in that a scalpel is as effective as a laser, and is less costly. The use of a laser to replace the function of a scalpel can also prove less effective for, and less popular among, surgeons. Use of a laser robs the device of its tactile feel, removing the element of feedback that many surgeons rely on to debride or cut precisely. Also, the removal of eschar, or heavy dead tissue, requires a large amount of energy to remove. It is impractical to use a laser for this function, when a mechanical means can prove as effective.

Debridement alone only removes dead tissue; it does nothing to promote healing directly. Debridement techniques are useful in cleaning a wound, thereby helping to prevent infection from bacteria present in the debrided tissue, but debridement alone will not prevent future infection or accelerate the healing process. However, used in conjunction with a healing technique, debridement can prove quite beneficial in achieving quick healing. Furthermore, wound healing techniques can be drastically improved by utilizing the benefits of debridement during radiation therapy.

There is a need to use sharp surgical debridement and high power electromagnetic photo-biostimulation in conjunction, to create an apparatus capable of the efficient removal of dead or infected wound tissue, while simultaneously destroying bacteria and viral agents, stimulating the growth of new tissue, and completing other processes to aid in wound healing. The present invention fills this need.

### OBJECTS AND BRIEF SUMMARY OF THE INVENTION

It is an object of this invention to provide a device and method that utilizes both sharp surgical and electromagnetic irradiation devices in conjunction for the debridement and healing of wounds.

It is another object of this invention to provide a device and method that applies non-ablative electromagnetic radiation of a sufficient power density over a sufficiently small period of time so as to effectively stimulate wound healing.

It is yet another object of this invention to provide a device and method that allows for the debridement, cleansing and healing of a wound and the removal or destruction of viral bodies and bacteria simultaneously or in rapid succession during a single procedure.

It is still another object of this invention to provide a device and method capable of delivering a means for electromagnetic irradiation of a wound with a penetration depth sufficient to destroy bacteria and viral agents and stimulate healing, but also shallow enough to avoid inflicting pain and further causing wounds.

It is a still further object of this invention to provide a device and method for promoting wound healing that is not only effective for otherwise healthy patients, but a device and method that is equally as effective for diabetic patients and patients with other conditions that inhibit natural wound healing.

Briefly stated, the present invention provides a method and device for wound debridement and wound healing that is equally effective for both diabetic and non-diabetic patients. A non-ablative laser beam or non-coherent radiation of a wavelength between 193 nm and 10.6 microns, preferably between 193 nm and 3 microns, is applied in combination with a mechanical or laser debriding apparatus. The radiation is delivered at a power density of at least approximately 1 W/cm² over a time period ranging from 1 second to 3 minutes. Typically, radiation power is in the range of 1 Watt to 15 Watts. Power density and treatment duration are adjusted to the parameters of the individual wound. Delivery means include, but are not limited to, optical fibers, articulating arms or direct exposure to radiation-emitting sources. A laser or mechanical scraping apparatus is used to remove dead or damaged tissue. Simultaneously, or in rapid progression, pulsed or continuous wave high power radiation of an appropriate wavelength is used to irradiate the exposed flesh. This device can be used on wounds of various depths to drastically cut down on the healing time by killing viral bodies and bacteria and performing other tasks such as vascularization.

The above, and other objects, features and advantages of the present invention will become apparent from the following description.

### DETAILED DESCRIPTION OF PREFERRED EMBODIMENTS

It has been found that the application of electromagnetic radiation at higher power and energy densities than previously used has a dramatic impact on the rate of wound healing for both diabetic and non-diabetic patients. The present invention, which can be used at a variety of wavelengths, appears to be equally successful for diabetic wound healing as it is for wound healing in otherwise healthy patients. In addition, it has also been found that higher power lasers in a range of wavelengths destroy bacteria and viral bodies, thus greatly reducing the risk of infection.

Means for delivering non-ablative laser or non-coherent radiation of a suitable tissue penetration depth is used in conjunction with a means for the debridement of wounds in humans or animals. A wavelength is chosen that will have a corresponding penetration depth of 0.1 mm to several mm, depending on the type of wound. Such a wavelength is in the range of 193 nm to 10.6 microns, and is preferably in the range from 193 nm to 3 microns. Said radiation is delivered with a power density of at least approximately 1 W/cm² over a predetermined treatment duration typically in the range of 1 second to 3 minutes. To achieve the desired power density over a sufficiently short period of time so as to effectively promote wound healing, radiation is typically delivered at a power between 1 Watt and 15 Watts, with an average power of 5-10 Watts. Although this power range is generally sufficient to achieve the desired energy density within a predetermined treatment duration, the present invention is not limited to this range. Parameters such as wavelength, energy density, power density and the use of continuous wave or pulsed radiation can be readily modified according to the characteristics of individual wounds.

The radiation described above is used to irradiate healthy exposed flesh, thus dramatically accelerating the healing process and simultaneously preventing infection by destroying any existing bacteria or viral bodies. With properly selected parameters, the radiation can penetrate deep enough to stimulate new growth and healing and to kill bacterial and viral bodies while remaining shallow enough to avoid overstimulating nerve endings in the skin which would cause pain or further damage.

In a preferred embodiment, radiation is utilized that will stimulate tissue and promote accelerated healing without heating the tissue and potentially causing damage. In this embodiment, a wavelength is chosen that is not overly absorbed in water. Because skin contains large amounts of water, a wavelength that is highly absorbed in water could thermally damage the skin. Accordingly, a wavelength between 193 nm and 1385 nm is chosen.

Electromagnetic radiation is delivered to the treatment site using known methods and devices. In one preferred embodiment, a laser or non-coherent beam or is “painted” onto the wound, or methodically scanned over the wound to achieve complete and consistent irradiation over the surface of the wound. Preferably, the beam is applied to the wound in a spiral pattern commencing at the edges of the wound and converging at the center. The spot size of the beam can be modified to achieve different power and energy densities to achieve different skin penetration depths. The delivery means can be a flat or shaped fiber, an optically transparent diamond or sapphire blade or cone, or any optical method that each wavelength could pass through. A collimating handpiece can also be utilized. Other embodiments include, but are not limited to, delivery of coherent radiation through an articulating arm, irradiation of the site through the use of light-emitting diodes, or delivery of non-coherent radiation using a lamp. Because only the radiation itself is of importance, a large variety of delivery means could be used, and thus the invention may not need a conventional delivery system.

The method involves scraping or otherwise debriding the damaged or infected tissue while simultaneously or intermittently irradiating the wound. Scraping or debriding devices comprise both laser and mechanical debridement means. The fiber used to deliver radiation for stimulating healing can be shaped to act both as a cutting or scraping tool and a radiation delivery apparatus. A mechanical scraping means, such as a scalpel, can be used as a separate device or attached to the radiation device. Additional fibers or other radiation means could be attached or used with the healing device to deliver a laser beam of sufficient energy density to act as a debridement tool by preferably ablating or vaporizing dead, damaged or diseased tissue. Alternatively, in another preferred embodiment, the energy density of the treatment laser can be intermittently modified to act as both a wound healing and an ablative device.

Another process by which the present invention promotes healing is by the removal of bacteria and undesired tissue by vaporizing moisture in the tissue. By this method, water or blood present in the wound site can be heated by means of suitable radiation. Sufficient heating causes the moisture to vaporize and thus expand, forcing harmful bacteria and unwanted tissue from the wound site. This process can be utilized as both a healing and a debridement tool. When used as a healing tool, the present invention removes bacteria, viruses or other contaminants with the vaporized moisture. This prevents infection and encourages faster healing.

As a debridement tool, radiation is utilized that will vaporize moisture and remove dead and damaged tissue from the wound. A method utilizing this process involves irradiating the treatment site with radiation of a suitable power and wavelength so that the radiation will penetrate the skin to a predetermined depth. The penetration depth is preferably chosen that it is equal to the depth at which damaged tissue and healthy tissue interface. Upon irradiation, moisture in the wound will expand and vaporize, forcing the damaged tissue away from healthy tissue and breaking up the damaged tissue. The broken up tissue may sluff off on its own or can be washed off or otherwise removed. In this manner, unwanted tissue is removed without the need for ablation or mechanical scraping. This method may be preferable over scraping or ablation, but there may be instances where ablation or mechanical debridement is preferred, such as for treatment of wounds where the wound depth is variable or uneven.

In a preferred embodiment, a device capable of delivering radiation with tunable power can be used to sufficiently modify the power density to alternately serve to promote healing processes and to both debride and heal by vaporizing moisture. This function can also be accomplished with a device capable of modifying the wavelength of radiation using a variety of fibers or light sources. The user can modify the wavelength to simultaneously or alternately accelerate healing, vaporize moisture for healing or debridement, and ablate unwanted tissue.

In one example of a preferred embodiment of the present invention, a method is described for alternating between irradiating the wound area to stimulate healing and debridement. Debridement can be accomplished either by a mechanical apparatus or by ablative or vaporizing radiation as described above. In this method, a discreet portion of the wound is debrided, followed by irradiation with a preselected wavelength and power density. A second portion is then treated in this way until the entire wound has been treated. This method is particularly suited for larger wounds, and is beneficial in that it allows the user to stimulate healing immediately after debridement and before possible contamination of the exposed tissue. A single device incorporating both means for stimulating healing and means for debriding is particularly useful in this instance. A single device allows the user to quickly and easily change from debridement to healing stimulation without interruptions in the procedure.

Other embodiments include the use of numerous fibers attached to the cutting apparatus, or emission of radiation via a lamp emitting non-coherent radiation of a specified wavelength or multiple wavelengths. Depending on the particular characteristics of the wound, different wavelengths may be more effective for the separate tasks of stimulating growth, stimulating collagen generation, killing bacteria, vascularization, cauterization, and healing stimulation. As a result, numerous fibers or combinations of different light sources may be needed to accomplish these tasks simultaneously.

The present invention is further illustrated by the following example, but is not limited thereby.

### EXAMPLE 1

The following is an example of a circular wound treated with a bare optical fiber with a 1000 micron diameter core. The parameters of the treatment are as follows:

| | | |
|---|---|---|
| Wavelength: | 980 | nm |
| Wound Area: | 12.56 | cm² |
| Spot size Area: | .636 | cm² |
| Power: | 5 | W |
| Treatment Duration: | 19.75 | s |
| Number of treatments: | 2 | |

The fiber is held 1 cm above the wound, and methodically scanned over the wound at a speed of 1 cm/s. The treatment duration is 19.75 seconds per treatment. The above parameters result in a total dose of energy of 198 Joules. The total energy density delivered to the wound is 16 J/cm² over two treatments, and the total power density (irradiance) is 0.8 W/cm², with a total treatment duration of 39.5 seconds.

The parameters of this example are illustrative only. These parameters may vary, depending on treatment time, wavelength used, radiation delivery device used, and number of treatments.

Example 2

An example of the effectiveness of the present invention is realized in a study done on the healing effects of high-powered laser treatment on mice, which demonstrates the effectiveness of high-powered laser wound treatment.

A group of 75 diabetic mice were used, which was divided into 5 subgroups of 15 mice, depending on the frequency of the treatment and power used. The five subgroups consisted of mice that received 980 nm laser treatment at 5 Watts every 2 days, 5 Watts every 4 days, 10 Watts every 2 days, 10 Watts every 4 days, and control group which were wounded but received no laser treatment. (Problem with 10 Watt: hair regrew around the edge of the wound, and when 10 W laser was applied, the hair caught fire. This may have affected the results of the 10 W treatment.)

The mice were anesthetized, and their dorsum shaved and cleansed. Then, two full-thickness circular wounds were created on the back of each mouse, using a skin punch biopsy instrument (6 mm diameter). Both wounds at either side of the spine received laser treatment according to the subgroup to which the mouse belonged. During laser treatment, the energy was applied for one second at a distance of approximately one centimeter from the wound. The wounds were then covered with Tegaderm (registered trademark of 3M Corporation) semi-transparent dressing for four days. Five mice from each subgroup, including the control group, were sacrificed at 1, 2 and 3 weeks.

Two perpendicular diameters of each wound were measured on the day of wounding and at days 5, 12 and 19, using digital calipers, and the area of the wounds were calculated. The percentage of wound closure was then calculated for each wound based on the total area of the wound. The following formula was used:

% wound closure on day x=(area on day x−area on day 0)/(area on day 0)×100

The results of average percentage of wound closure and average percentage of wounds closed at the time of sacrifice for the diabetic mice are shown below.

| | % Wound Closure | | | % of Wounds Healed | | |
|---|---|---|---|---|---|---|
| | Day 5 | Day 12 | Day 19 | Day 5 | Day 12 | Day 19 |
| 5W × 2d | 39.0 | 83.5 | 100.0 | 0.0 | 11.1 | 100.0 |
| 5W × 4d | 25.3 | 71.5 | 91.2 | 0.0 | 0.0 | 30.0 |
| Control | 37.9 | 81.9 | 91.5 | 0.0 | 0.0 | 25.0 |

The results indicate that the wounds from all of the diabetic groups, including the control group, were reduced in size over time. The wounds that received 5 Watts of power every 2 days appeared to close the fastest. In addition, some of the wounds were completely healed by day 12, and all wounds were healed by day 19. Those receiving 5 Watts every 4 days seemed to close at a rate similar to the controls, but a greater percentage of the wounds were healed completely after 19 days. Thus, treatment of diabetic ulcers at 5 Watts appears to enhance healing.

Example 3

The following is an example of a preferred treatment method, which can be used to treat a foot or lower leg ulcer. In this example, the patient receives weekly treatments for up to eight weeks or until the wound is completely or substantially closed.

Upon arrival of the patient, a physical examination of the affected area is performed, assessing characteristics such as protective sensation in the foot and vascular status. The depth and surface area of the wound is measured. The wound is then cleansed and laser treatment is commenced.

In this suggested method, a collimating handpiece is used in conjunction with a mechanical debriding apparatus such as a scalpel. Laser energy is applied in a 90-degree crosshatch pattern to insure complete wound coverage. An energy density of 18 J/cm² is used, and applied using variable power settings and treatment times as prescribed in the following graph. These power settings and treatment times prescribed in the graph and the energy density of 18 J/cm2 were selected because, based on the findings from a preliminary study, laser energy can be applied at this level without causing tissue damage or without the need for local anesthetic.

[image]

By way of illustration, the following calculation is based on a wound area of 12 cm2 irradiated with a laser emitted from a collimated handpiece with a power setting of 5 Watts. The treatment time to be used is calculated from the following equation:

(Wound Area×Treatment Density)/(Power Setting×Handpiece Efficiency)=Time of treatment (seconds)

Inserting the proper values yields the following treatment time:

(12 cm2×18 J/cm2)/(5 Watts×0.75)=(216 J)/(3.75 W)=57.6 seconds

Therefore, the wound area should be treated for a total of 57.6 seconds, or 0.96 minutes, to achieve optimal healing enhancement without damage to the treatment area or the need for anesthetic.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A device for wound debridement and healing comprising means for wound debridement integrated with means for delivering non-ablative electromagnetic radiation for wound healing.

2. A device for wound debridement and healing according to claim 1, wherein said means for delivering non-ablative electromagnetic radiation is at least one optical fiber optically connected to a source of non-ablative electromagnetic radiation.

3. A device for wound debridement and healing according to claim 1, wherein said non-ablative electromagnetic radiation operates at one or more wavelengths in a range from 193 nm to 10.6 μm.

4. A device for wound debridement and healing according to claim 3, wherein said non-ablative electromagnetic radiation operates at one or more wavelengths in a range from 193 nm to 3 μm.

5. A device for wound debridement and healing according to claim 3, wherein said non-ablative electromagnetic radiation is provided by a laser.

6. A device for wound debridement and healing according to claim 3, wherein said non-ablative electromagnetic radiation is provided by a non-coherent source.

7. A device for wound debridement and healing according to claim 1, wherein said means for delivering non-ablative electromagnetic radiation is a collimating delivery means optically connected to a source of non-ablative electromagnetic radiation.

8. A device for wound debridement and healing according to claim 1, wherein said wound debridement means comprises a mechanical scraping apparatus.

9. A device for wound debridement and healing according to claim 1, wherein said wound debridement means comprises a means for delivering electromagnetic radiation with a sufficient power to ablate/remove dead, diseased and damaged tissue.

10. A device for wound debridement and healing according to claim 1, wherein said device is capable of simultaneously irradiating a treatment site with radiation of more than one wavelength.

11. A device for wound debridement and healing according to claim 1, wherein said wound debridement means and said means for delivering non-ablative electromagnetic radiation are employable simultaneously.

12. A method for wound debridement and healing comprising the steps of:
a. debriding a wound with a wound debridement means; and
b. irradiating said wound with non-ablative electromagnetic radiation.

13. A method for wound debridement and healing according to claim 12, wherein debriding and irradiating steps occur intermittently.

14. A method for wound debridement and healing according to claim 12, wherein debriding and irradiating steps occur simultaneously.

15. A method for wound debridement and healing according to claim 12, wherein said irradiating step uses non-ablative electromagnetic radiation having a power density of at least about 1 W/cm² for a preselected time of exposure in a range from 1 second to 3 minutes.

16. A method for wound debridement and healing according to claim 12, wherein said irradiating step uses non-ablative electromagnetic radiation operating at one or more wavelengths in a range from 193 nm to 10.6 μm.

17. A method for wound debridement and healing according to claim 16, wherein said irradiating step uses non-ablative electromagnetic radiation operating at one or more wavelengths in a range from 193 nm to 3 μm.

18. A method for wound debridement and healing according to claim 12, further comprising a preliminary step of setting a radiation source for said irradiating step to operate in one of two modes: a continuous wave mode and a pulsed mode.

19. A method for wound debridement and healing according to claim 15, wherein said irradiating step uses non-ablative radiation having an average power between 1 W and 20 W.

20. A method for wound debridement and healing according to claim 19, wherein said irradiating step uses non-ablative radiation having an average power between 5 and 10 W.

21. A method for wound debridement and healing according to claim 12, wherein said irradiating step also includes destroying bacteria and viral bodies, thereby preventing infection.

22. A method for wound debridement and healing according to claim 12, wherein said irradiation step uses non-ablative electromagnetic radiation to stimulate vascularization and collagen generation for wound healing in both diabetic patients and non-diabetic patients.

23. A method for wound debridement and healing according to claim 12, wherein said irradiating step involves applying said non-ablative radiation in a spiral pattern, starting at an edge and converging on a center of said wound.

24. A method for wound debridement and healing according to claim 12, wherein said debriding step uses a mechanical scraping apparatus.

25. A method for wound debridement and healing according to claim 12, wherein said debriding step uses an apparatus for delivering electromagnetic radiation with sufficient power to ablate/remove dead, diseased and damaged tissue.
